(19) United States
(12) Patent Application Publication
(10) Pub. No.: US 2005/0277827 A1
(43) Pub. Date: Dec. 15, 2005

(54) Pleural fluid localization device and method of using

(76) Inventor: Alexandre Carvalho, Dallas, TX (US)

Correspondence Address: National IP Rights Center, LLC;Suite 400, 550 Township Line Road, Blue Bell, PA, 19422, US

(21) Appl. No.: 10/864,762

(22) Filed: Jun. 9, 2004

(51) Int. Cl. A61B 5/05

(52) U.S. Cl. 600/414

(57) ABSTRACT

A novel pleural fluid localization device and method of using is disclosed. The device includes a marker, having a top side, a bottom side adapted to be affixed to the body of a patient, a top edge, a first side edge, a second side edge, and a bottom edge; and a first plurality of markings on the marker to guide a clinician in localizing an area on the body a patient to perform a procedure on the patient. The method includes placing the marker on the body of the patient; imaging the patient; reading the location on the plurality of markings on the marker to localize the procedure on the patient; and performing the procedure on the patient at the location read on the plurality of markings on the marker during the step of reading the location on the plurality of markings on the marker to localize the procedure on the patient.

### FIELD OF THE INVENTION

The present invention is directed to the field of pleural draining devices and pleural nodule detection from CT thoracic images. In particular, the present invention is directed to a novel concept of a measuring tape placed against the chest wall skin to localize pleural fluid.

### BACKGROUND OF THE INVENTION

The present invention is directed to a device to be used in aiding the localization of pleural fluid or pleural based anomalies in anticipation of a diagnostic invasive procedure of the chest. The device is a measuring tape, similar to a ruler with radio opaque markings. It is placed like a strip of tape against the chest wall skin during a routine postero anterior Chest X-Ray (CSR) or CT. By comparing the position of a pleural abnormality against the measuring tape position, a clinician may find the best and safest area to perform a biopsy, tap or chest tube placement.

U.S. Patent Application No. 2003/0099389 discloses an algorithm that recovers regions of possible pleural nodules left out of an organ field or otherwise undetected due to the nature of low level image processing in the organ field. A morphological closing with an elliptical structuring element is performed on a region to detect nodules within the size of the ellipsoid. A deformable surface-based analysis is performed in distinctive regions for the identification of larger nodules. The integrated use of a deformable surface model and chamfer distance potential enables explicit representation of regularized, or smoothed, surfaces within which nodule candidates may be detected.

U.S. Pat. No. 5,431,633 discloses a method and a noninvasive device for preventing pressure build-up in pleural space and reducing possibility of development of tension pneumothorax from open pneumothorax by providing a rigid, tubular base firmly attached to an annular flange and a noninvasive, flexible, pliable sheet with a central aperture for closing a sucking chest wound of a patient, while providing a possible path for discharging air, body fluids or any combination thereof from the pleural space of a patient. A generally flat, one-way check valve for allowing air discharge is located in the tubular base. A protective, tubular cover comprises a screening means and is used for protecting the check valve from foreign debris while allowing exit of air, body fluids or any combination thereof from the check valve and enabling visual monitoring of fluid levels within the device.

U.S. Pat. No. 6,254,581 discloses a device useful for draining fluid from the pleural cavity of a patient includes two enclosed chambers. The first chamber has at its upper end a one-way directional valve. The valve, which is provided with a connector for connection with a catheter draining fluid from the patient's pleural cavity, operates only to allow fluid to flow into the first chamber. The lower end of the first chamber, which is releasably connected to the second chamber, is optionally provided with a second one-way directional valve that operates only to allow fluid to flow from the first chamber into the second chamber. The second enclosed chamber of the device of the invention has an upper end and a closed lower end. The upper end of the second chamber is releasably connected to the lower end of the first chamber to provide a substantially fluid-tight connection. The device is provided with an outlet to the ambient atmosphere; the outlet can be disposed in either the first or the second enclosed chamber.

U.S. Pat. No. 5,344,410 discloses a device comprising an outer tube having a central canal and perforations at its one end, a stylet fitted in the central canal of the outer tube, an inner tube, which is in fact a non-return valve and with its one end tightly connected to the outer tube and with its opposite end stretched over the stylet with a possibility of slipping off the latter and getting everted. The device has a receptacle for collecting the pleural fluid, tightly connected to the outer tube and the inner tube, and a retainer for fixing the device in position.

None of the above inventions, however, allow for a clinician to localize an area on the body where the clinician can perform a medical procedure such as a tap or biopsy, among other things. It would therefore be beneficial if a device and method existed that would allow a clinician to localize an area on the patient's body where a procedure such as a tap, biopsy, or the like should be performed.

### OBJECTS AND SUMMARY OF THE INVENTION

It is an object of the present invention to provide a device and method to localize on a patient where a procedure is to be performed.

It is a further object of the present invention to provide a pleural fluid localization device that includes a marker, having a top side, a bottom side adapted to be affixed to the body of a patient, a top edge, a first side edge, a second side edge, and a bottom edge; and a first plurality of markings on the marker to guide a clinician in localizing an area on the body a patient to perform a procedure on the patient.

It is yet a further object of the present invention to provide a method of using a pleural fluid localization device having a plurality of markings on the marker to localize an area on the body of a patient for a clinician to perform a procedure that includes the steps of placing the marker on the body of the patient; imaging the patient; reading the location on the plurality of markings on the marker to localize the procedure on the patient; and performing the procedure on the patient at the location read on the plurality of markings on the marker during the step of reading the location on the plurality of markings on the marker to localize the procedure on the patient.

In accordance with a first aspect of the present invention, a novel pleural fluid localization device is provided. The novel pleural fluid localization device includes a marker, having a top side, a bottom side adapted to be affixed to the body of a patient, a top edge, a first side edge, a second side edge, and a bottom edge; and a first plurality of markings on the marker to guide a clinician in localizing an area on the body a patient to perform a procedure on the patient.

In accordance with another aspect of the present invention, a novel method of using a pleural fluid localization device having a plurality of markings on the marker to localize an area on the body of a patient for a clinician to perform a procedure is provided. The method includes the steps of placing the marker on the body of the patient; imaging the patient; reading the location on the plurality of markings on the marker to localize the procedure on the patient; and performing the procedure on the patient at the location read on the plurality of markings on the marker during the step of reading the location on the plurality of markings on the marker to localize the procedure on the patient.

### BRIEF DESCRIPTION OF THE DRAWINGS

The foregoing summary, as well as the following detailed description of a preferred embodiment of the present invention will be better understood when read with reference to the appended drawings, wherein:

FIG. 1A is a top plan view of a pleural fluid localization device in accordance with the present invention.

FIG. 1B is a partial perspective view of the pleural fluid localization device of FIG. 1A.

FIG. 1C is a plurality of top plan views of the pleural fluid localization device of FIG. 1A.

FIG. 2 is a schematic depiction of the pleural fluid localization device shown positioned on a patient for x-ray.

FIG. 3A is a posterior view in partial cross section of an imaging slide having the pleural fluid localization device positioned on the patient.

FIG. 3B is a side elevation view in partial cross section of an imaging slide having the pleural fluid localization device positioned on the patient.

FIG. 4A is a schematic depiction of a patient having a pleural fluid tap at a position determined by a clinician reading the pleural fluid location device.

FIG. 4B is a top plan view in partial cross section of an imaging slide having the pleural fluid localization device positioned on the patient.

### DETAILED DESCRIPTION OF THE PREFERRED EMBODIMENT

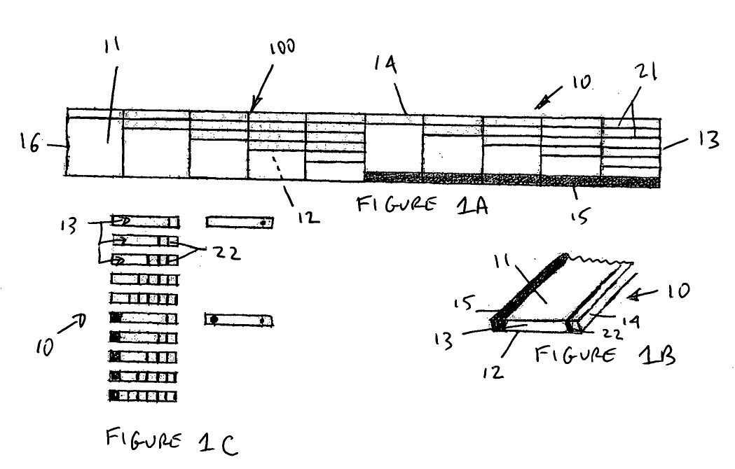

Referring now to the drawings wherein like reference numerals refer to like components across the several views, and in particular to FIGS. 1A, 1B, and 1C, there is shown a pleural fluid location device 10. The marker 100 includes a marker 100, having a top side 11, a bottom side 12, a top edge 13, a bottom edge 16, a first side edge 14, and a second side edge 15.

A first plurality of markings 21 are included on the top side 11 of the marker 100. In a preferred embodiment of the present invention, the first plurality of markings 21 are a plurality of lines on the top side 11 of the marker 100, however any type of markings known to one of ordinary skill in the art may be utilized as the first plurality of markings 21. A second plurality of markings 22 are included on the top edge 13 of the marker 100. In a preferred embodiment of the present invention, the second plurality of markings 22 are a plurality of lines on the top edge 13 of the marker 100, however any type of markings known to one of ordinary skill in the art may be utilized as the second plurality of markings 22. The bottom side 12 of the pleural fluid location device 10 is adapted to be affixed to the body of a patient, for example via an adhesive. However, any known means to affix the pleural fluid location device 10 to the body of the patient known to one of ordinary skill in the art may be employed.

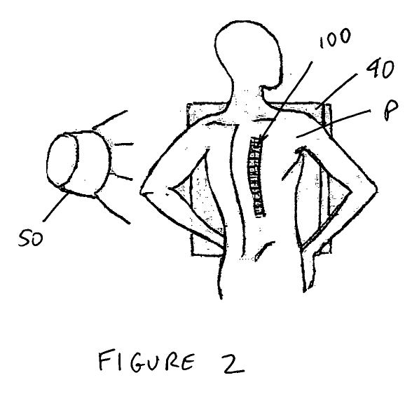

Referring now to FIG. 2, the marker 100 is shown placed on the back of a patient P. The patient P is depicted as standing in front of an imaging screen 40, and in between an imager 50 and the imaging screen 40. In a preferred embodiment of the present invention, the imaging screen 40 and the imager 50 are part of an x-ray imaging device, however any imaging device known to one of ordinary skill in the art may be employed as the imaging device, including a CT scanner.

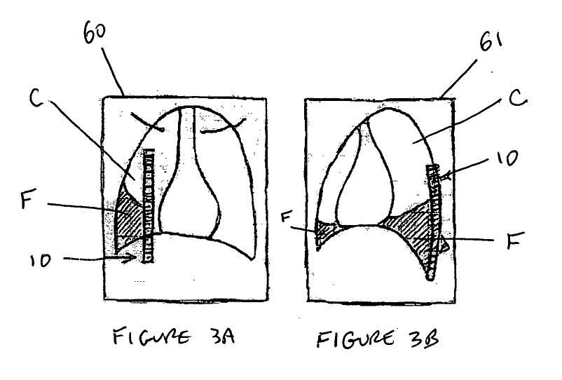

Referring now to FIGS. 3A and 3B, an imaging slide 60 and an imaging slide 61 are shown. The imaging slide 60 shows a chest cavity C in partial cross section from the posterior view, with the marker 100 on the body of the patient P. The imaging slide 61 shows the chest cavity C in partial cross section from the side elevation view, with the marker 100 on the body of the patient P. In both the imaging slides 60 and 61, a location of pleural fluid F can be seen in the chest cavity C. The pleural fluid F in the chest cavity C is easily located with regards to the first plurality of markings 21 on the top side 11 of the marker 100. Specifically, the first plurality of markings 21 allow a clinician to localize the pleural fluid F in the chest cavity C along the top side of the marker 100.

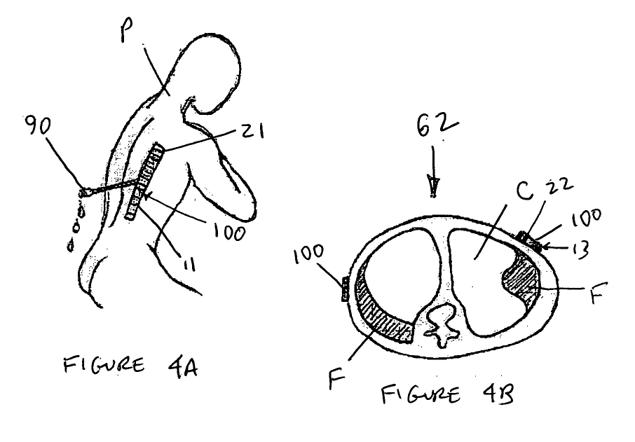

Referring now to FIG. 4B, a top plan view in partial cross section of and imaging slide 62 of the chest cavity C of the patient P is shown. The pleural fluid F in the chest cavity C is easily located with regards to the second plurality of markings 22 on the top edge 13 of the marker 100. Specifically, the second plurality of markings 22 allow the clinician to localize the pleural fluid F in the chest cavity C along the top edge 13 of the marker 100. In a preferred embodiment of the present invention, the first plurality of markings 21 and the second plurality of markings 22, respectively, are spaced at even intervals.

Referring now to FIGS. 2, 3A, 3B, 4A, and 4B, a preferred method of using the marker 100 to localize an area on the body of the patient P for the clinician to perform a procedure, such as a fluid tap in the chest cavity C, will be described. First the clinician places the marker 100 on the patient P. The clinician then takes an image of the patient P, and reads the slides 60, 61, and 62 in order to determine where the pleural fluid F is located within the chest cavity C. Specifically, the clinician notes the location of the pleural fluid F relative to the first plurality of markings 21 on the top side 11 of the marker 100, and the location of the pleural fluid F relative to the second plurality of markings 22 on the top edge 13 of the marker 100. The clinician then performs the appropriate procedure at the location determined by the first plurality of markings 21 and the second plurality of markings 22. For example, the clinician may perform a fluid tap using an instrument 90 by inserting the instrument 90 at the location of the pleural fluid F in the chest cavity C as located in relation to the first plurality of markings 21 and the second plurality of markings 22.

In view of the foregoing disclosure, some advantages of the present invention can be seen. For example, a novel pleural fluid localization device is disclosed. The novel pleural fluid localization device allows for a clinician to localize the area where he or she is to perform a procedure on a patient. In this way, the clinician can be accurate the first time and not have to make multiple attempts at performing the procedure.

While the preferred embodiment of the present invention has been described and illustrated, modifications may be made by one of ordinary skill in the art without departing from the scope and spirit of the invention as defined in the appended claims. For example, in a preferred embodiment of the present invention, the imaging system may be an x-ray device, however any imaging device known to one of ordinary skill in the art may be utilized as the imaging device. Additionally, the procedure in a preferred embodiment of the present invention may be a fluid tap to tap fluid from the chest cavity, however any procedure that could be performed, known by one of ordinary skill in the art may be performed at the location determined by using the pleural fluid localization device, including, among other things, a chest tube insertion. Removal of a tissue sample for biopsy is another useful application of the present invention.

1. A pleural fluid localization device, comprising:
a marker, having a top side, a bottom side adapted to be affixed to the body of a patient, a top edge, a first side edge, a second side edge, and a bottom edge; and
a first plurality of markings on the marker to guide a clinician in localizing an area on the body a patient to perform a procedure on the patient.

2. The device of claim 1, wherein the first plurality of markings are located on the top side of the marker.

3. The device of claim 2, wherein the first plurality of markings are spaced at even intervals on the top side of the marker.

4. The device of claim 1, further comprising a second plurality of markings on the marker to guide a clinician in localizing an area on the body a patient to perform a procedure on the patient.

5. The device of claim 4, wherein the second plurality of markings on the marker are located on the top edge of the marker.

6. The device of claim 5, wherein the second plurality of markings are spaced at even intervals on the top side of the marker.

7. A method of using a pleural fluid localization device having a plurality of markings on the marker to localize an area on the body of a patient for a clinician to perform a procedure, comprising the steps of:
placing the marker on the body of the patient;
imaging the patient;
reading the location on the plurality of markings on the marker to localize the procedure on the patient; and
performing the procedure on the patient at the location read on the plurality of markings on the marker during the step of reading the location on the plurality of markings on the marker to localize the procedure on the patient.

8. The method of claim 7, wherein the step of imaging the patient includes the step of imaging the patient using an X-Ray machine.

9. The method of claim 7, wherein the step of imaging the patient includes the step of imaging the patient with a CT scanner.

10. The method of claim 7, wherein the marker having a plurality of markings includes a top side, bottom side, top edge, bottom edge, first side edge, and second side edge, and the step of reading the location on the plurality of markings on the marker to localize the procedure on the patient includes reading a first set of markings which are located on the top side of the marker.

11. The method of claim 7, wherein the marker having a plurality of markings includes a top side, bottom side, top edge, bottom edge, first side edge, and second side edge, and the step of reading the location on the plurality of markings on the marker to localize the procedure on the patient includes reading a second set of markings which are located on the top edge of the marker.

12. The method of claim 7, wherein the step of performing the procedure on the patient at the location read on the plurality of markings on the marker during the step of reading the location on the plurality of markings on the marker to localize the procedure on the patient includes inserting a tap to drain fluid from the location.

13. The method of claim 7, wherein the step of performing the procedure on the patient at the location read on the plurality of markings on the marker during the step of reading the location on the plurality of markings on the marker to localize the procedure on the patient includes obtaining a sample for biopsy from the location.

14. The method of claim 7, wherein the step of performing the procedure on the patient at the location read on the plurality of markings on the marker during the step of reading the location on the plurality of markings on the marker to localize the procedure on the patient includes placing a chest tube at the location.
